(19) United States
(12) Patent Application Publication
(10) Pub. No.: US 2017/0181881 A1
(43) Pub. Date: Jun. 29, 2017

(54) PLATE FOR SAGITTAL CORRECTION OF THE SPINE

(71) Applicant: Hvidovre Hospital, Hvidovre (DK)

(72) Inventors: Christian Wong, Kgs. Lyngby (DK); Jan Nielsen, Faxe Ladeplads (DK)

(73) Assignee: Hvidovre Hospital, Hvidovre (DK)

(21) Appl. No.: 15/325,261

(22) PCT Filed: Jul. 10, 2015
(86) PCT No.: PCT/DK2015/050214

§ 371 Date: Jan. 10, 2017

(30) Foreign Application Priority Data
Jul. 15, 2014 (DK) PA 2014 70448

(51) Int. Cl. A61F 5/02

(52) U.S. Cl. CPC A61F 5/026; A61F 5/028

(57) ABSTRACT

The present disclosure relates to ascoliosis monopiece plate, to fit tightly on the back of a user, comprising a lower section and an upper section, wherein the upper section is rounded in the sagittal plane, such that the plate is configured to force the thoracic spine of the user to bend forward in the sagittal plane. The disclosure furthermore relates to a method for manufacturing a scoliosis monopiece plate, to fit tightly on the back of a user, comprising a lower section and an upper section, wherein the upper section is rounded in the sagittal plane, such that the plate is configured to force the thoracic spine of the user to bend forward in the sagittal plane and a method for reducing risk and/or treating spinal deformities, such as scoliosis, of a patient by applying pressure to the thoracic spine of the patient, such that the thoracic spine of the patient is bent forward in the sagittal plane.

The present invention relates to a scoliosis monopiece plate for sagittal correction of the spine, a method for manufacturing a plate for sagittal correction of the spine, and a method for reducing risk and/or treating spinal deformities of a patient.

### BACKGROUND OF INVENTION

Scoliosis is a medical condition in which a person's spine is curved from side to side. The definition of scoliosis is a spinal curvature to the right or left in the coronal plane.

Idiopathic adolescent scoliosis (AIS) has been described as early as 400 BC by Hippocrates. Since then, numerous studies have been conducted to clarify the aetiopathology behind AIS, suggesting a broad variety from genetic, growth, chemical, biomechanical to neuromuscular causes. So far no single causal theory for AIS has prevailed and it is still a conundrum and regarded as a multifactorial disease.

The conventional medical management of scoliosis for children and adolescents include observation, bracing and surgery. For moderate scoliosis, orthotic spinal bracing is the traditional treatment, sometimes combined with an exercise program. Bracing is normally done when the patient has bone growth remaining. In general, the coronal plane deformity is corrected by pressuring the spine ‘straight’ from sides of the trunk and preventing the curve from progressing to the point where surgery is recommended.

The effectiveness of these braces has been questioned by some clinicians and researchers since the published evidence to support it is inconclusive. Most of the known braces are not able to provide a lasting true correction of the spinal deformities. Most of the braces are adapted to apply relatively high counter pressure to fixate the deformities and may be uncomfortable to wear. In particular the braces with hard plastic modules, such as the Boston brace, may be relatively heavy and warm for the user to wear. These braces often demand 18 to 23 hours of daily wear through skeletal maturity.

### SUMMARY OF INVENTION

The present invention relates to a scoliosis monopiece plate to be placed on the back of the user. The plate treats the cause of the scoliosis rather than the medical condition that is the consequence. The theoretical basis for the functioning of the plate is that AIS is developed as a consequence of the spine growing into a mechanically unstable column and seems to progress in a self-sustaining ‘vicious cycle’ due to the effect of gravity and asymmetric loads in a growth-modulating buckling-like manner. In this regard adolescent females distinguish themselves generally from males by a significantly increased and earlier thoracic growth. These factors coincide with a temporal sagittal flattening of thoracic vertebrae for adolescent females. The sagittal flattening contributes to the overall rotational instability of the spine. Therefore, in order to counteract this rotational instability, the present invention reduces the sagittal flattening of thoracic vertebrae. The present invention relates to a monopiece plate that is shaped to fit tightly on the back of a user. The upper section of the plate is rounded in the sagittal plane, such that the plate is configured to force the thoracic spine of the user to bend forward in the sagittal plane, thereby creating an external hyperkyphosing thoracic posture.

As mentioned one advantage of the disclosed plate is that it treats the cause of the scoliosis rather than the medical condition that is the consequence. Another advantage is that the plate can be made significantly smaller and lighter than for example a traditional Boston brace, thus more comfortable to wear. The plate is placed directly on the back of the user and is preferably supported by a supplementary soft brace or clothes. The soft brace may have different shapes and materials but is preferable designed to give solid support for the monopiece plate, and at the same time have a light, discrete appearance. The soft brace may be made of for example a thin, elastic material, wrapped around the torso and/or shoulders. It should be remembered that the plate (or a traditional brace) is to be worn during a significant portion of the day.

The present invention also relates to a method for manufacturing a scoliosis monopiece plate, to fit tightly on the back of a user, comprising a lower section and an upper section, wherein the upper section is rounded in the sagittal plane. The inventors have realized that the process of manufacturing an individually fitted monopiece plate can be automatized to some extent. The method comprises the steps of creating a three-dimensional image of the back of the user (for example by scanning and/or photographing and/or drawing and/or casting the back of the user), creating a three-dimensional image of a plate based on the three-dimensional image of the back, and finally forming a scoliosis monopiece plate according to the three-dimensional image of the plate (for example by carving, cutting, grinding or molding). The upper section of the plate should, additionally, be bent in the sagittal plane. This can be achieved either by (virtually) bending the upper section of the plate of the image or by bending the upper section of the (physical) monopiece plate.

### DESCRIPTION OF DRAWINGS

The invention will in the following be described in greater detail with reference to the accompanying drawings. The drawings are exemplary and are intended to illustrate some of the features of the present method and unit and are not to be construed as limiting to the presently disclosed invention.

FIG. 1 shows a front view of a monopiece plate, configured to force the thoracic spine of the user to bend forward in the sagittal plane, wherein the upper section is divided in the longitudinal direction into a number of sub-sections.

FIG. 2 shows a front view of a monopiece plate, configured to force the thoracic spine of the user to bend forward in the sagittal plane, wherein the upper section is divided in the longitudinal direction into a number of sub-sections, with spacing between the sub-sections.

FIG. 3 shows a side view of a monopiece plate next to a user, said plate configured to force the thoracic spine of the user to bend forward in the sagittal plane, wherein the upper section of the plate is rounded in the sagittal plane.

### DEFINITIONS

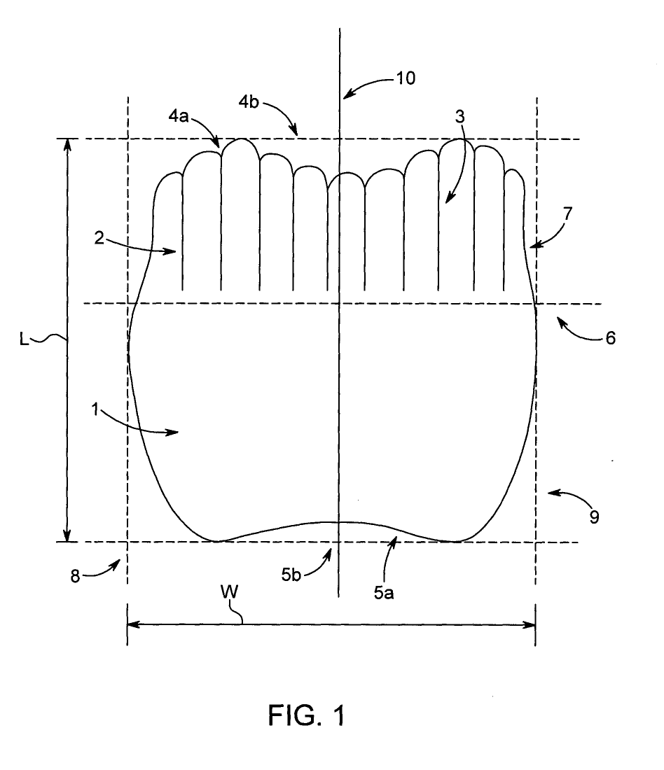

Upper section is defined as the upper part of the plate when the plate is attached on the back of the user. Hence, it is the upper section of the plate that is rounded in the sagittal plane to provide the force that bends the thoracic spine of the user forward in the sagittal plane. FIG. 1 shows an example of a monopiece plate wherein the boundary 6 divides the plate in an upper and a lower section.

Lower section is defined as the lower part of the plate when the plate is attached on the back of the user. FIG. 1 shows an example of a monopiece plate wherein the boundary 6 divides the plate in an upper and a lower section.

Upper edge is defined as the upper edge of the upper section of the plate when the plate is attached on the back of the user. Hence, the upper edge is the edge closest to the head of the user when the plate is attached on the back of the user.

Lower edge is defined as the lower edge of the lower section of the plate when the plate is attached on the back of the user.

Upper horizontal extension of the upper edge is defined as a line horizontally extended from the upmost point on the upper edge of the plate. Horizontal is defined as transversal to a vertical central axis of the plate.

Lower horizontal extension of the lower edge is defined as a line horizontally extended from the lowest point on the lower edge of the plate.

Length is defined as the distance between the upper horizontal extension of the upper edge and the lower horizontal extension of the lower edge of the plate. In FIG. 1 the length of the plate is marked ‘L’.

Width is defined as the distance between the side edge extensions of the plates. In FIG. 1 the width of the plate is marked ‘W’ and is the distance between the side edge extensions 8 and 9.

Sagittal plane is defined as a vertical plane which passes from anterior to posterior of the body, dividing the body into right and left halves.

Coronal plane (also known as the frontal plane) is any vertical plane that divides the body into ventral and dorsal (belly and back) sections.

### DETAILED DESCRIPTION OF THE INVENTION

The presently disclosed invention relates to a scoliosis monopiece plate, to fit tightly on the back of a user, comprising a lower section and an upper section, wherein the upper section is rounded in the sagittal plane, such that the plate is configured to force the thoracic spine of the user to bend forward in the sagittal plane. Conventional brace treatment has been aimed at treating the apparent coronal plane deformity, wherein the coronal plane deformity is corrected by pressuring the spine ‘straight’ from sides of the trunk. Examples of conventional braces include for example the Boston brace and the Providence brace. The development of scoliosis is typically associated with adolescent females. Principally, AIS has developed as a consequence of the spine growing into a mechanically unstable column and seems to progress in a self-sustaining ‘vicious cycle’ due to the effect of gravity and asymmetric loads in a growth-modulating buckling-like manner. The spinal growth spurt for both sexes coincides with adolescence, but adolescent females distinguish themselves from males by a significantly increased and earlier thoracic growth. These factors coincide with a temporal sagittal flattening of thoracic vertebrae for adolescent females. This contributes to the overall rotational instability of the spine. One aspect of the presently disclosed invention is therefore to treat the primary cause of the condition rather than the consequence. By forcing thoracic spine of the user to bend forward in the sagittal plane, a kyphosis effect of thoracic spine is achieved, which counteracts the rotational instability. The presently disclosed monopiece plate is shaped to fit tightly on the back of a user; however, the rounded upper section of the plate is further bent slightly forward to create a force on thoracic vertebrae, thereby creating an external hyperkyphosing thoracic posture. The plate according to the present invention is shaped to fit tightly on the back of a user, more precisely to an external area corresponding to the position of thoracic vertebrae, which, as stated above, is also the target to bend. However, the plate may also extend beyond the upper back in all directions in order to achieve optimal support. The plate is typically placed on the upper back of the user to create a pressure on the upper part of thoracic vertebrae. However, a portion of the plate may be configured to be placed on the lower back of the user. The reason for doing this is typically to target the sagittal flattening of the lower part of the thoracic spine. This means that the purpose is still the same, i.e. to bend the thoracic spine forward in the sagittal plane, but the bending is performed where it makes most sense based on the shape of the spine of the user. The main advantage of the disclosed plate is that it treats a cause of the scoliosis rather than the medical condition that is the consequence. Another advantage is that the plate can be made significantly smaller and lighter than for example a traditional Boston brace, thus more comfortable to wear. In a preferred embodiment of the present invention, the plate is placed directly on the back of the user, typically supported by a supplementary soft brace or clothes, which is further explained below. The plate may furthermore be adapted specifically to one user, or, in another embodiment, produced to fit a group of users. The latter can be achieved by having a number of mass-producible sizes and shapes of the plate. Shape in this context has a broad meaning, including the general shape of the plate that makes the plate fit on the back and feel comfortable for the user, the roundness of the rounded upper section and the angle in which the upper section is bent forward.

Support Element(s)

In order to keep the plate at a fixed position on the back of the user, supplementary support is typically needed, preferably clothes or a supplementary soft brace that holds the plate in a fixed position. Such a soft brace may be designed to have a discrete appearance, for example a thin, soft material wrapped around the torso, neck, arm(s) or shoulder(s). It may cover the presently disclosed plate, not only to give support but also to hide it. The supporting soft brace may also comprise strap(s), belt(s) or band(s) to tighten and fixate the plate. The bands may be elastic and could be made of for example silicon. The support could also be designed as clothing, thereby reducing the need for using both external support and clothes. In one embodiment the soft brace comes as a part of the presently disclosed plate, i.e. the plate further comprises a soft brace configured to hold the plate. Both the supporting soft brace and clothing may be made of an elastic material, such as spandex. The advantage of using such a material for supporting the plate is that it is well suited for holding the plate in a fixed position and still comfortable to wear. In one embodiment the soft brace is configured to provide proprioceptive pressure feedback to the user. Proprioception is the sense of the relative position of neighbouring parts of the body and strengths of effort being employed in the movement. In this context the proprioceptive pressure from the soft brace can help the user to vary muscle contraction and thereby stabilize the spine. In one embodiment the supplementary soft brace is configured to de-rotate thoracic vertebrae around the axis of said thoracic vertebrae. The soft brace is configured to pull the upper back of the user to the left or to the right to achieve a de-rotation of thoracic vertebrae in order to counteract the spinal rotation that is associated with scoliosis. By pulling the upper back to the left or right, thereby counteracting the spinal rotation, the soft brace can assist in holding the spine in a more neutral position

In one embodiment of the presently disclosed plate the clothing, strap, band or belt is configured to further adjust the bending of the thoracic spine in the sagittal plane. As stated, these elements can be made of e.g. an elastic material. They do not necessarily have the function of holding the plate in a fixed position—they could also be employed to assist the plate in forcing the thoracic spine of the user to bend forward in the sagittal plane. These extra elements can be considered to be elements for adjusting the force on the thoracic spine. In one embodiment the clothing, strap, band or belt is/are adjusted based on information from visual indicator(s) on the plate and/or stretch sensor(s) and/or bend sensor(s). This can be done either directly by an integrated mechanism, or indirectly by a means for processing the measured data and controlling and adjusting a mechanism for changing either the place directly or the assisting clothing/strap/band. In one embodiment there is unit connected to indicators and/or stretch and/or bend sensors. The user can then either evaluate the measured data or adjust the plate and/or clothing/strap/band manually or by means of a control unit.

A further aspect of the presently disclosed invention relates to the plate being configured to be supported externally by a hard brace. Such a hard brace may be wrapped around the body of the user, and it may be configured to apply pressure to the spine from sides of the trunk. This embodiment is particularly advantageous for users that would like to combine the main advantage of the present invention (treating the cause of the scoliosis by bending the thoracic spine in the sagittal plane and thereby reduce the rotational instability) with the conventional way of dealing with the lateral deformity by pressuring the spine ‘straight’ from sides of the trunk.

Shape

As mentioned, the rounded section of the plate, which is the upper section of the plate, should be further bent forward in the sagittal plane in order to achieve a kyphosis effect of thoracic spine. ‘Further bent forward’ in this context shall be interpreted as the rounded section being bent more than the upper back without the plate attached. This is what creates the force on thoracic vertebrae. The area 3 on the upper back of the user in FIG. 1 is an example of where the force can be applied to achieve a kyphosis effect of thoracic spine. In this regard the lower section of the plate can be seen as a fixed section, and in relation to this fixed section the upper rounded section should be bent between 3° and 15° forward in the sagittal plane, for example 3°, 4°, 5°, 6°, 7°, 8°, 9°, 10°, 11°, 12°, 13°, 14° or 15°. The bending angle should be chosen such that a certain pressure is created on thoracic vertebrae.

Thoracic vertebrae compose the middle segment of the vertebral column, between the cervical vertebrae and the lumbar vertebrae. In humans, there are twelve thoracic vertebrae, T1-T12. The kyphotic angle is defined as the kobb angle between T1 and T12, 25-40° typically being considered normal. Because of temporary sagittal flattening of thoracic vertebrae, adolescent females may have a curve that measures less than the normal values. In the presently disclosed invention, the rounded section of the plate may be shaped such that the curvature corresponds to a predefined kyphotic angle of thoracic vertebrae between 5° and 25°, for example 5°, 6°, 7°, 8°, 9°, 10°, 11°, 12°, 13°, 14°, 15°, 16°, 17°, 18°, 19°, 20°, 21°, 22°, 23°, 24° or 25°. By shaping the rounded section of the plate after a preferred kyphotic angle rather than exactly after the actual kyphotic angle of the user, the external pressure that is to create a kyphosis effect of thoracic spine can be achieved.

In one embodiment of the presently disclosed invention the upper section constitutes between 25% and 75% of the total area of the plate, such as between 30% and 70%, or such as between 35% and 65%, or such as between 40% and 60% of the total area of the plate, for example 25%, 26%, 27%, 28%, 29%, 30%, 31%, 32%, 33%, 34%, 35%, 40%, 45%, 50%, 55%, 55%, 60%, 65%, 70%, or 75%.

As mentioned, one aspect of the presently disclosed invention relates to the plate being light and comfortable to wear. In order for the plate to fit on the back of the user, the length of the plate should be in the range of 200 mm-500 mm, such as in the range of 250 mm-450 mm, or such as in the range of 250 mm-400 mm, for example 200 mm, 205 mm, 210 mm, 215 mm, 220 mm, 230 mm, 240 mm, 250 mm, 260 mm, 270 mm, 280 mm, 290 mm, 300 mm, 350 mm, 400 mm, 450 mm, or 500 mm.

The lower section is preferably shaped to fit tightly on the lower part of the upper back of the user, but may also extend to the lower back of the user. The lower section is typically substantially straight, but may also be slightly curved to fit the upper or lower back.

Another aspect of the presently disclosed invention relates to the rounded section of the place being elastic. If the section that creates the pressure on thoracic vertebrae is elastic, the pressure is maintained even if the user moves the back such that it temporarily does not have the normal shape. To some extent it also means that the plate does not have to be manufactured as precisely as a plate without elasticity. This means that the plate can be mass-produced in a number of predefined sizes and shapes, which may be used by patients without a need to have the exact measurement of the individual.

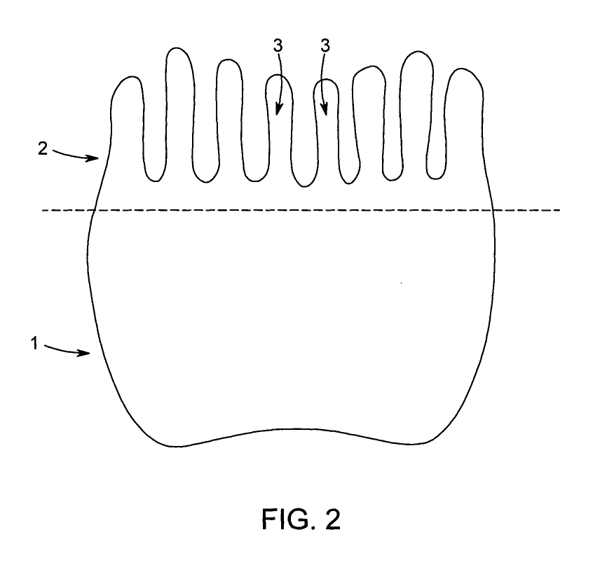

In another embodiment, the rounded section is divided in the longitudinal direction into a predefined number of elastic sub-sections. This is another advantage related to flexibility of the section of the plate that provides the pressure on thoracic vertebrae. By splitting the rounded section into a number of sub-sections, wherein each sub-section can contribute independently, a more flexible plate is obtained in terms of distributing the total force evenly. There may be spacing between the sub-sections, as illustrated in FIG. 2.

In one embodiment of the present invention the lower edge of the plate is configured to be placed such that it rests on the pelvic bone. One reason for doing this is that the force on thoracic vertebrae is greater with a longer lever, and the pelvic bone constitutes a suitable lower limit for the plate. This part of the plate could potentially also help reducing the lumbar lateral instability by providing support to the lumbar spine. Alternatively, the lower edge of the plate is configured to be placed above the pelvic bone, resulting in a shorter lever but a lighter plate. The upper edge of the plate is typically configured to be placed below the cervical vertebrae.

In another embodiment of the present invention, the plate further comprises a rigid support element extending from the lower edge of the plate, wherein the support is configured to rest on the pelvic bones. This can be seen as an alternative to having a plate that rests directly on the pelvic bone. The support element can be made of a material suitable for giving support and can be designed to give maximum support from the pelvic bone.

Materials

A shape-memory alloy (SMA, smart metal, memory metal, memory alloy, muscle wire, smart alloy) is an alloy that ‘remembers’ its original shape and that when deformed returns to its pre-deformed shape when heated. In one embodiment of the invention the plate is made of shape-memory alloy, such as Cu—Al—Ni, Ni—Ti, Fe—Mn—Si or Cu—Zn—Al. Such a material in the present invention is beneficial since it means that the plate can be adjusted for a tight fit on the back of the user every time it is attached. After use the plate may return to its original shape.

It is important that the plate is light since it may be worn for longer periods. Therefore the plate is preferably made of a light material, selected from a group of plastics, such as polyethylene, polypropylene, polycarbonate (PC) or polymethylmethacrylate (PMMA). However, other may also be used. Preferably, the plate is made as thin in order to provide a discrete appearance. Furthermore, the plate can be painted in an individual colour, chosen by the user.

In one embodiment of the present invention the plate further comprises a soft coating, such as yarn, polyurethane-coated textile, temperature responsive textile, or any textile. The soft coating has the purpose of making the plate more comfortable to wear.

Referencing

In one embodiment of the present invention the plate further comprises at least one reference element for indicating a bending angle between the upper section and the lower section. It is important that the plate has the correct angle when the user wears the plate. Since there is a counter-force from the body of the user when the plate is worn, it may be useful to know the actual bending angle and other angles when the force of the plate and the counter-force of the user's body are applied. In one embodiment the reference elements are visual indicators, such as drawn lines, marks or similar on the plate. Alternatively, there may be stretch and/or bend sensors attached to the plate to indicate the bending angle, either as a an angle, or as an angle in relation to a starting point.

Electric Stimulation

In a further embodiment the plate further comprises an integrated electric stimulation element. Alternatively, the electric stimulation element can be a separate element. Electrical stimulation should be construed to include a range of purposed such as transcutaneous electrical nerve stimulation (TENS) or electrical muscle stimulation (EMS). In a preferred embodiment the electric stimulation is transferred to skin by means of pads, such as gel pads, configured to be attached to the skin. In one embodiment the electric stimulation element(s) is/are connected to a stimulation control unit for controlling the at least one electric stimulation element.

Logging and Activity Monitoring

Since correcting scoliosis is typically a long process, the present disclosure further relates to the logging and monitoring of a number of parameters, some of which can be considered to be relatively long term. It may be required, or at least beneficial, to wear the plate a substantial part of the day. In one embodiment the plate further comprises a logging element and/or an activity monitoring element. The logging data can for example collecting information regarding the daily use, which can be used by medical staff to evaluate the progress of the scoliosis. The data can also be used for statistical analysis, for example by evaluating on a number of patients how different wearing patterns or configurations of the plate correlates with the progress of the scoliosis. Preferably, the scoliosis itself is also be measured and monitored.

Manufacturing

The present invention also relates to a method for manufacturing a scoliosis monopiece plate, to fit tightly on the back of a user, comprising a lower section and an upper section, wherein the upper section is rounded in the sagittal plane, such that the plate is configured to force the thoracic spine of the user to bend forward in the sagittal plane. The method comprises the steps of:
creating a three-dimensional image of the back of at least one user;
creating a three-dimensional image of a plate based on the three-dimensional image of the back;
forming a scoliosis monopiece plate according to the three-dimensional image of the plate.

The inventors have realised that the process of manufacturing a monopiece place can be automatized to some extent. In order to manufacture an individually fitted plate it is necessary to have the physical measures of the back of the user. The first step of the method is to create a three-dimensional image of the back of the user. For this purpose a 3D-scanner can be used. Another option is to provide a number of photos from different angles, which can be translated by a machine to a three-dimensional image of the back of the user. The back may also be photographed with x-rays. Based on the three-dimensional image of the back it is possible to create an image (i.e. a virtual model) of the plate to be manufactured. Based on the image of the plate it is then possible to manufacture the plate, for example by carving, cutting, grinding or molding.

This process may be automatic or manual. As mentioned the plate should be configured to force the thoracic spine of the user to bend forward in the sagittal plane. This can be achieved by (virtually) bending the upper section of the plate of the image between 3° and 15°, for example 3°, 4°, 5°, 6°, 7°, 8°, 9°, 10°, 11°, 12°, 13°, 14° or 15°, forward in the sagittal plane in relation to a fixed lower section of the plate of the image, or by (virtually) bending the three-dimensional image of the plate such that the curvature of the upper section of the plate of the image corresponds to a predefined kyphotic angle of thoracic vertebrae between 5° and 25°. Alternatively, it can be achieved by bending the upper section of the monopiece plate between 3° and 15°, for example 3°, 4°, 5°, 6°, 7°, 8°, 9°, 10°, 11°, 12°, 13°, 14° or 15°, forward in the sagittal plane in relation to a fixed lower section of the monopiece plate, or by bending the upper section of the monopiece plate, such that the curvature corresponds to a predefined kyphotic angle of thoracic vertebrae between 5° and 25°. Manufacturing of the monopiece plate such that it fits a group of users is also possible. This enables that the plate can be mass-produced in a number of predefined sizes and shapes based on the image of the back of at least one user as described above.

Treating Spinal Deformities

An additional aspect of the presently disclosed invention relates to a method for treating spinal deformities, such as scoliosis, of a patient by applying pressure to thoracic spine of the patient, thereby forcing the thoracic spine of the patient to bend forward in the sagittal plane. The method may be used on mild scoliosis with a spinal curvature of a few degrees to the right or left in the coronal plane, but also on scoliosis of more than 10 degrees to the right or left in the coronal plane. The method preferably uses the monopiece plate described in the present disclosure. As explained above, the effectiveness of conventional braces, wherein the coronal plane deformity is corrected by pressuring the spine ‘straight’ from sides of the trunk, has been questioned by some clinicians and researchers since the published evidence to support it is inconclusive. By applying pressure to thoracic spine of the patient, thereby forcing the thoracic spine of the patient to bend forward in the sagittal plane, an external hyperkyphosing thoracic posture is created, which counteracts the rotational instability of the spine. As a consequence, over time, in particular if the treatment is started early, the AIS is stabilised in both the thoracic and lumbar segment.

### EXAMPLES

FIG. 1 shows a front view of an embodiment of a monopiece plate, configured to force the thoracic spine of the user to bend forward in the sagittal plane. The plate is divided into a lower section 1 and an upper section 2 by a boundary 6. The boundary 6 does not have a technical function other than defining the boundary between the upper and lower section of the plate. 4a is the upper edge of the plate and 4b is the upper horizontal extension of the upmost point on the upper edge 4a (drawn as a horizontal dotted line). Upper in this context should be interpreted as in relation to a line transversal to the central axis 10. In this case it can be seen that the contour of the upper edge 4a is not the same as the upper horizontal extension 4b (horizontal dotted line). Similarly there is a lower edge 5a and a lower horizontal extension 4b. The upper section is divided in the longitudinal direction into a number of sub-sections 3. 7 in this example illustrates a shape of the plate that allows the arm of the user to move. Side edge extension 8 and 9 are extensions of the leftmost and rightmost points of the side edges of the plate. The extensions are parallel to the central axis 10.

FIG. 2 shows a front view of a monopiece plate similar to the plate in FIG. 1, configured to force the thoracic spine of the user to bend forward in the sagittal plane, wherein the upper section is divided in the longitudinal direction into a number of sub-sections, with spacing between the sub-sections, comprising a lower section 1 and an upper section 2. In this example there is spacing between the sub-sections 3.

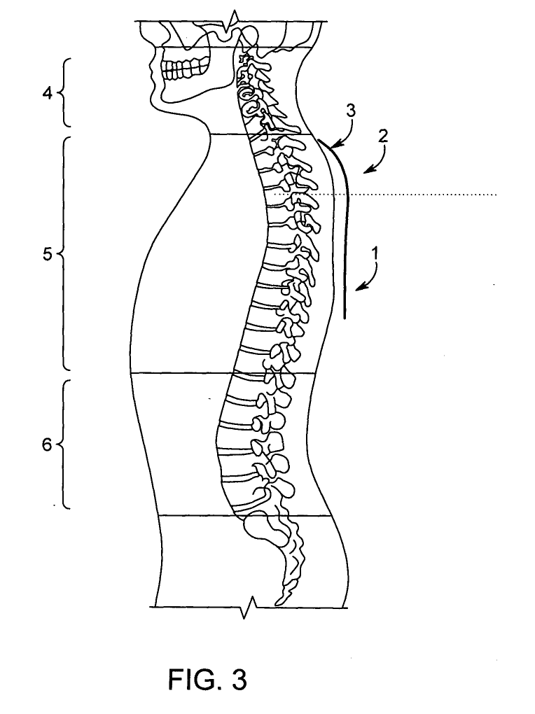

FIG. 3 shows a side view of a monopiece plate next to a user, comprising a lower section 1 and an upper section 2. The rounded upper part forces the thoracic spine of the user to bend forward in the sagittal plane by providing a force at the area 3 of the upper back. 4, 5, and 6 indicate the cervical vertebrae, thoracic vertebrae and lumbar vertebrae respectively. The plate is placed at the upper part of thoracic vertebrae and is configured to force the thoracic spine of the user to bend forward in the sagittal plane.

### FURTHER DETAILS OF THE INVENTION

The invention will now be described in further detail with reference to the following items:
1. A scoliosis monopiece plate, to fit tightly on the back of a user, comprising a lower section and an upper section, wherein the upper section is rounded in the sagittal plane, such that the plate is configured to force the thoracic spine of the user to bend forward in the sagittal plane.
2. The plate according to any of the preceding items, wherein the plate is configured to be placed on the upper back of the user.
3. The plate according to any of the preceding items, wherein a portion of the plate is configured to be placed on the lower back of the user.
4. The plate according to any of the preceding items, wherein the upper section constitutes between ¼ and ¾ of the total area of the plate.
5. The plate according to any of the preceding items, wherein the upper section is divided in the longitudinal direction into at least two sub-sections.
6. The plate according to item 5, wherein there is spacing between at least two of the sub-sections.
7. The plate according to item 5, wherein the sub-sections are elastic in the sagittal plane, independently of each other.
8. The plate according to any of the preceding items, wherein the upper section is elastic.
9. The plate according to any of the preceding items, wherein the upper section is made of shape-memory alloy, such as Cu—Al—Ni, Ni—Ti, Fe—Mn—Si or Cu—Zn—Al.
10. The plate according to any of the preceding items, further comprising at least one reference element for indicating a bending angle between the upper section and the lower section.
11. The plate according to item 10, wherein the reference element is/are visual indicator(s) on the plate and/or stretch sensor(s) and/or bend sensor(s).
12. The plate according to any of the preceding items, wherein the upper section is bent between 3° and 15° forward in the sagittal plane in relation to a fixed lower section of the plate.
13. The plate according to any of the preceding items, wherein the upper section is shaped such that the curvature corresponds to a predefined kyphotic angle of thoracic vertebrae between 5° and 25°.
14. The plate according to any of the preceding items, said plate having a width between 200 mm and 500 mm.
15. The plate according to any of the preceding items, wherein the lower edge of the plate is configured to be placed such that said lower edge rests on the pelvic bone.
16. The plate according to any of the preceding items, wherein the lower edge of the plate is configured to be placed above the pelvic bone.
17. The plate according to any of the preceding items, wherein said plate is made of a light material, selected from a group of plastics, such as polyethylene, polypropylene (PP), polycarbonate (PC), polymethylmethacrylate (PMMA) or Acrylonitrile butadiene styrene (ABS).
18. The plate according to any of the preceding items, further comprising a soft coating.
19. The plate according to any of the preceding items, wherein the plate is adapted to fit a group of users.
20. The plate according to any of the preceding items, further comprising a rigid support element extending from the lower edge of the plate, wherein the support is configured to rest on the pelvic bones.
21. The plate according to any of the preceding items, configured to be supported externally by clothing, and/or at least one soft brace and/or at least one strap, and/or at least one band, and/or at least one belt.
22. The plate according to any of the preceding items, further comprising clothing, and/or at least one soft brace and/or at least one strap, and/or at least one band, and/or at least one belt, configured to hold the plate.
23. The plate according to item 22, wherein the clothing and/or the at least one soft brace is configured to provide proprioceptive pressure feedback to the user.
24. The plate according to any of items 22-23, wherein the clothing, and/or the at least one soft brace and/or the at least one strap, and/or the at least one band, and/or the at least one belt is made of spandex.
25. The plate according to any of items 22-24, wherein the bands are elastic and/or the bands are made of silicon.
26. The plate according to any of items 22-25, wherein the soft brace and/or clothing is configured to rotate thoracic vertebrae around the axis of said thoracic vertebrae.
27. The plate according to any of items 22-26, wherein the clothing, strap, band or belt is configured to further adjust the bending of the thoracic spine in the sagittal plane.
28. The plate according to item 27, wherein the clothing, strap, band or belt is/are adjusted based on information from the visual indicator(s) on the plate and/or stretch sensor(s) and/or bend sensor(s).
29. The plate according to any of items 27-28, further comprising a bending control unit for controlling and adjusting the clothing, strap, band or belt.
30. The plate according to any of the preceding items, configured to be supported externally by a hard brace.
31. The plate according to item 30, wherein the hard brace is wrapped around the body of the user.
32. The plate according to any of items 30-31, wherein the hard brace is configured to apply pressure to the spine of the user from sides of the trunk of the user.
33. The plate according to any of the preceding items, further comprising at least one electric stimulation element integrated in the plate.
34. The plate according to item 33, wherein the at least one electric stimulation element is/are pads, preferably gel pads, configured to be attached to the skin.
35. The plate according to any of items 33-34, further comprising a stimulation control unit for controlling the at least one electric stimulation element.
36. The plate according to any of the preceding items, further comprising a logging element and/or an activity monitoring element.
37. The plate according to item 36, wherein the logging element and/or activity monitoring element is/are configured to log and/or the wearing times by the user and/or status regarding the scoliosis.
38. A method for manufacturing a scoliosis monopiece plate, to fit tightly on the back of a user, comprising a lower section and an upper section, wherein the upper section is rounded in the sagittal plane, such that the plate is configured to force the thoracic spine of the user to bend forward in the sagittal plane, comprising the steps of:
   creating a three-dimensional image of the back of at least one user;
   creating a three-dimensional image of a plate based on the three-dimensional image of the back;
   forming the scoliosis monopiece plate according to the three-dimensional image of the plate.
39. The method according to item 38, wherein the three-dimensional image is obtained by scanning and/or photographing and/or drawing and/or casting and/or x-raying the back of the user.
40. The method according to any of items 38-39, wherein the three-dimensional image of the plate is modified such that the upper section of the plate of the image is bent between 3° and 15° forward in the sagittal plane in relation to a fixed lower section of the plate of the image.
41. The method according to any of items 38-39, further comprising the step: bending the upper section of the monopiece plate between 3° and 15° forward in the sagittal plane in relation to a fixed lower section of the monopiece plate.
42. The method according to any of items 38-39, wherein the three-dimensional image of the plate is modified such that the curvature of the upper section of the plate of the image corresponds to a predefined kyphotic angle of thoracic vertebrae between 5° and 25°.
43. The method according to any of items 38-39, further comprising the step: bending the upper section of the monopiece plate, such that the curvature corresponds to a predefined kyphotic angle of thoracic vertebrae between 5° and 25°.
44. The method according to any of items 38-41, wherein the monopiece plate is carved, cut, grinded or molded.
45. The method according to any of items 38-44, wherein the monopiece plate is adapted to fit a group of users.
46. A method for reducing risk and/or treating spinal deformities, such as scoliosis, of a patient by applying pressure to the thoracic spine of the patient, such that the thoracic spine of the patient is bent forward in the sagittal plane.
47. The method according to item 46, using the plate according to any of items 1-32.

1.-20. (canceled)

21. A scoliosis monopiece plate, comprising:
a plate having an upper section extending to an upper edge and a lower portion extending to a lower edge, a length of the plate being defined as a distance between an uppermost point on the upper edge and a lowermost point on a lower edge;
the plate being adapted to fit tightly on the back of a user such that the upper edge of the upper section is disposed in a region of the thoracic vertebra of the user;
the upper section of the plate being bent forwardly in a sagittal plane of the user such that the upper edge is forward of a remainder of the upper section and applies a force on the thoracic vertebra of the user, thereby forcing the thoracic spine of the user to bend forward in the sagittal plane.

22. A scoliosis monopiece plate according to claim 21, further comprising a supplementary support adapted hold the plate and to fit the plate tightly on the back of the user.

23. A scoliosis monopiece plate according to claim 21, wherein the upper section of the plate is bent between 3° and 15° forward in the sagittal plane in relation to a fixed lower section of the plate or the upper section of the plate is shaped such that the curvature corresponds to a predefined kyphotic angle of thoracic vertebrae between 5° and 25°.

24. A scoliosis monopiece plate according to claim 21, wherein the upper section is divided in a longitudinal direction into at least two sub-sections and/or at least two of the sub-sections are spaced apart from each other so as to provide a spacing therebetween.

25. A scoliosis monopiece plate according to claim 24, wherein the sub-sections are elastic in the sagittal plane, independently of each other.

26. A scoliosis monopiece plate according to claim 21, wherein the upper section is elastic.

27. A scoliosis monopiece plate according to claim 21, wherein the upper section is bent forwardly more than the upper back in the region of the thoracic vertebra of a user is bent forward when the plate is not fitted to the back of the user.

28. A scoliosis monopiece plate according to claim 21, wherein the upper section of the plate constitutes between ¼ and ¾ of a total area of the plate.

29. A scoliosis monopiece plate according to claim 21, wherein the plate has a width between 200 mm and 500 mm.

30. A scoliosis monopiece plate according to claim 21, wherein the plate is made of a light material, selected from a group of plastics consisting of polyethylene, polypropylene (PP), polycarbonate (PC), polymethylmethacrylate (PMMA) and Acrylonitrile butadiene styrene (ABS).

31. A method for reducing risk of and/or treating spinal deformities and/or scoliosis of a patient, comprising:
providing a scoliosis monopiece plate, the plate having an upper section extending to an upper edge and a lower portion extending to a lower edge, the upper section of the plate being rounded forwardly in a sagittal plane such that the upper edge is forward of a remainder of the upper section;
fitting the scoliosis monopiece plate tightly on the back of the patient with the upper edge positioned in the thoracic region;
applying force on thoracic vertebra of the patient with the upper edge of the monopiece plate, thereby forcing the thoracic spine of the user to bend forward in the sagittal plane.

32. A method according to claim 31, wherein the fitting step comprising fitting an entirety of the monopiece plate on the upper back of the patient.

33. A method according to claim 31, further comprising providing a supplementary support adapted to hold the plate, the step of fitting the scoliosis monopiece plate tightly on the back of the patient comprising securing the plate tightly on the back using the supplementary support.

34. A method according to claim 33, wherein the supplementary support is selected from the group consisting of clothing, at least one soft brace, at least one strap, at least one band, and at least one belt.

35. A method according to claim 33, further comprising the step of adjusting the fit of the monopiece plate using the supplemental support based on a reference element.

36. A method according to claim 33, further comprising the step of adjusting the fit of the monopiece plate using the supplemental support to adjust the bending of the thoracic spine in the sagittal plane.

37. A method according to claim 31, wherein the upper section of the plate is bent forwardly more than the upper back in the region of the thoracic vertebra of a user is bent forward when the plate is not fitted to the back of the user.

38. A method according to claim 31, further comprising the step of applying electrical stimulation to the skin of the patient and/or logging activity of the patient.

39. A method according to claim 31, further comprising manufacturing the scoliosis monopiece plate, comprising the steps of:
creating a three-dimensional image of the back of at least one patient;
creating a three-dimensional image of a plate based on the three-dimensional image of the back; and
forming the scoliosis monopiece plate according to the three-dimensional image of the plate.

40. A method according to claim 31, wherein the fitting step further comprises positioning the plate such that the lower edge of the plate is on the pelvic bone or such that the lower edge of the plate is above the pelvic bone.
